(19) United States
(12) Patent Application Publication
(10) Pub. No.: US 2016/0066603 A1
(43) Pub. Date: Mar. 10, 2016

(54) FULVIC ACID AND DERIVATIVES AND METHODS OF PREPARATION AND USE

(71) Applicant: EMPIRE TECHNOLOGY DEVELOPMENT LLC, Wilmington, DE (US)

(72) Inventors: Georgius Abidal ADAM, Edensor Park (AU); Scott Andrew NEEDHAM, Mangerton, New South Wales (AU)

(21) Appl. No.: 14/785,566

(22) PCT Filed: Apr. 18, 2013
(86) PCT No.: PCT/US13/37144

§ 371 Date: Oct. 19, 2015

(51) Int. Cl. A23L 1/03; A23L 1/304; C07D 311/42; C07C 65/11; C07C 323/62; C07C 62/32

(52) U.S. Cl. CPC A23L 1/0315; C07C 323/62; C07C 62/32; C07D 311/42; C07C 65/11; A23L 1/3045; A23L 1/304; C07C 2101/16; C07C 2102/10

(57) ABSTRACT

Disclosed are antioxidative, natural compounds, their salts, chelates and cleavage derivatives that exhibit a superior combination of properties. The compounds can be used for a variety of purposes, including stabilizing foods, cosmetics, beverages and nutritional supplements. The compounds can be prepared by hydrolyzing a fulvic acid of formula I or VIII to provide at least one antioxidant compounds of formula II, formula III, formula IV, formula V, formula VI, formula VII, salts, or chelates thereof.

### BACKGROUND

Various types of antioxidant additives are used to protect food from oxidation, inhibiting the effect of oxygen in the atmosphere such as using soluble vitamin C (ascorbic acid and its salts with sodium, calcium, etc.) to protect some fruits or meat, or ascorbic stearate or palmitate as oil or fat soluble antioxidants for use with other foods. Currently used anti-oxidants include BHT (butylated hydroxytoluene), TBHQ (t-butyl hydroquinone), BHA (butylated hydroxy anisole), gallic acid, and gallic esters.

Some natural antioxidants, such as vitamins, minerals, and enzymes, in addition to their antioxidant activity, are also regarded as nutrients due to their bioactivity. Ascorbic acid (Vitamin C) and tocopherols (a class of compounds with Vitamin E activity) are the most important commercial natural antioxidants but they are expensive and unstable in processing and storage at high temperatures.

### SUMMARY

New antioxidants for use in food processing are preferably nontoxic, inexpensive, effective at low concentration (0.001-0.02%), surviving processing, stable in finished products, devoid of undesirable colour, flavour, and odour effects, and most importantly be compatible with food products. Fulvic acid has been reported as a human nutrition supplement in a liquid form as a component of a mineral colloid formula.

This disclosure details inexpensive food preservatives based on fulvic acid, fulvic acid derivatives, derivative salts and chelates. These compounds are stable, non-toxic, have high bioavailability, and are suitable for both oil and water based foods due to good solubility in both water and oil. Modification of the fulvic acid structure for use as a food stabilizer and in forming natural anti-oxidant based food packaging materials is described herein.

Hydrolysis of fulvic acid may be used to synthesize its cleavage derivatives, salts, and chelates by alkaline hydrolysis under controlled conditions. The anti-oxidation efficiency of these derivatives is due to their chemical structure which contains all the functional groups known as antioxidants and free radical scavenger active groups. The fulvic acid and fulvate derivatives demonstrate high efficiency as an anti-oxidant, have highly stable activities, are highly nutritious, and have tremendous potential as food additives as well as anti-oxidant/thermal stabilizers for various polymer systems, particularly food-safe polymeric packaging.

Embodiments thus provide methods for stabilizing foods, beverages, cosmetics and/or nutritional supplements by the application of fulvic acid hydrolysis derived compounds or compositions, in an amount sufficient to have a measurable stabilizing effect.

Other embodiments further provide stabilized foods, beverages, cosmetics and/or nutritional supplements comprising a food, beverage, cosmetic and/or nutritional supplement, together with a stabilizing composition consisting of antioxidants derived from hydrolysis of fulvic acid.

Embodiments include methods for stabilizing the fresh flavor and preventing the formation of off-flavors in a food product matrix, such as in a dairy product, fat, oil, fat emulsion, edible ice, fruit, vegetable, fungi, seaweed, nuts, seeds, confectionery, cereal, cereal product derived from cereal grains, bakery ware, meat, meat byproduct, fish, fish product, egg product, sugar, artificial sweetener, spices, condiment, soup, sauce, salad, protein mix, non-dairy beverage, or savory snack. The food products are treated at some stage in their production with an effective amount of an antioxidant composition derived from hydrolyzed fulvic acid; in a manner which does not adversely impact the taste or color of the foods.

Other embodiments provide methods for stabilizing the fresh flavor and color and preventing the formation of off-flavors and off-colors in a food product matrix such as a vegetable oil, animal fat, processed cheeses, chewing gum base, processed meat products, dried meats, sausages, beef patties, meatballs, frozen seafood, pizza toppings, protein, yeast, bakery products, dry cereals, spices, dehydrated potatoes, potato chips, beverage mixes, nonalcoholic beverages, mixed nuts, fruit, vegetables, butter, margarine, dairy products, and the like, by incorporating into these materials at some stage in their production, an effective amount of one or more antioxidant compounds derived from hydrolysis of fulvic acid.

### DESCRIPTION OF DRAWINGS

FIG. 1: A thermo-oxidation DSC (Differential Scanning calorimetry) thermogram for unstabilized low density polyethylene; sample size 16.5 mg, 5° C.-300° C. at 5° C./minute, DSC Instrument 2920MDSC V2.6A.

FIG. 2: A thermo-oxidation DSC thermogram for low density polyethylene stabilized by 0.1% hydrolyzed fulvic acid; sample size 11.5 mg, 5° C.-300° C. at 5° C./minute, DSC Instrument 2920MDSC V2.6A.

FIG. 3: A thermo-oxidation DSC thermogram for low density polyethylene stabilized by 0.5% hydrolyzed fulvic acid; sample size 15.1 mg, 5° C.-300° C. at 5° C./minute, DSC Instrument 2920MDSC V2.6A.

### DETAILED DESCRIPTION

The above summary of the present technology is not intended to describe each illustrated embodiment or every possible implementation of the present technology. The detailed description, which follows, particularly exemplifies these embodiments.

Before the present compositions and methods are described, it is to be understood that they are not limited to the particular compositions, methodologies or protocols described, as these may vary. It is also to be understood that the terminology used in the description is for the purpose of describing the particular versions or embodiments only, and is not intended to limit their scope which will be limited only by the appended claims.

It must also be noted that as used herein and in the appended claims, the singular forms “a”, “an”, and “the” include plural reference unless the context clearly dictates otherwise. Unless defined otherwise, all technical and scientific terms used herein have the same meanings as commonly understood by one of ordinary skill in the art. Although any methods and materials similar or equivalent to those described herein can be used in the practice or testing of embodiments disclosed, the preferred methods, devices, and materials are now described.

“Optional” or “optionally” means that the subsequently described event or circumstance may or may not occur, and that the description includes instances where the event occurs and instances where it does not.

“Substantially no” means that the subsequently described event may occur at most about less than 10% of the time or the subsequently described component may be at most about less than 10% of the total composition, in some embodiments, and in others, at most about less than 5%, and in still others at most about less than 1%.

Antioxidative compounds, their salts, and chelates useful for stabilizing foods, cosmetics, beverages and nutritional supplements can be prepared from fulvic acid by hydrolysis under controlled conditions. The anti-oxidation efficiency of these sustainable natural derivatives is due to their chemical structure which contains functional groups that are known as antioxidants and free radical scavenger active groups.

The chemical structure of a fulvic acid (I) has hydrolysable ester groups.

The structure of a model fulvic acid is illustrated by formula (VIII).

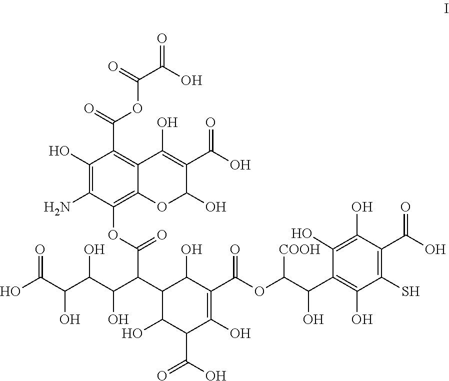 [SMILES: Nc1c(O)c(C(=O)OC(=O)C(=O)O)c2c(c1OC(=O)C(C(O)C(O)C(O)C(=O)O)C1C(O)C(C(=O)OC(C(=O)O)C(O)c3c(O)c(O)c(C(=O)O)c(S)c3O)=C(O)C(C(=O)O)C1O)OC(O)C(C(=O)O)=C2O]

Hydrolysis of ester bonds of fulvic acid I generates compounds II-VII. Compounds I-VIII are active multi pH buffers since they dissolve at all pH values. The cleavage derivatives of fulvic acid I at the three ester groups, its carboxylate salts and chelates have lower molecular weight than fulvic acid I. These derivatives also have high oxygen content due to carboxylate groups adjacent to carbonyl or hydroxyl groups. The chemical compounds I-VIII demonstrate highly stable activities and are highly nutritious. The low molecular weight structures derived demonstrate a higher efficiency as anti-oxidants than fulvic acid I, and the chemical compounds I-VIII have tremendous potential as food additives as well as anti-oxidant/thermal stabilizers for various polymer systems, particularly food-safe polymeric packaging.

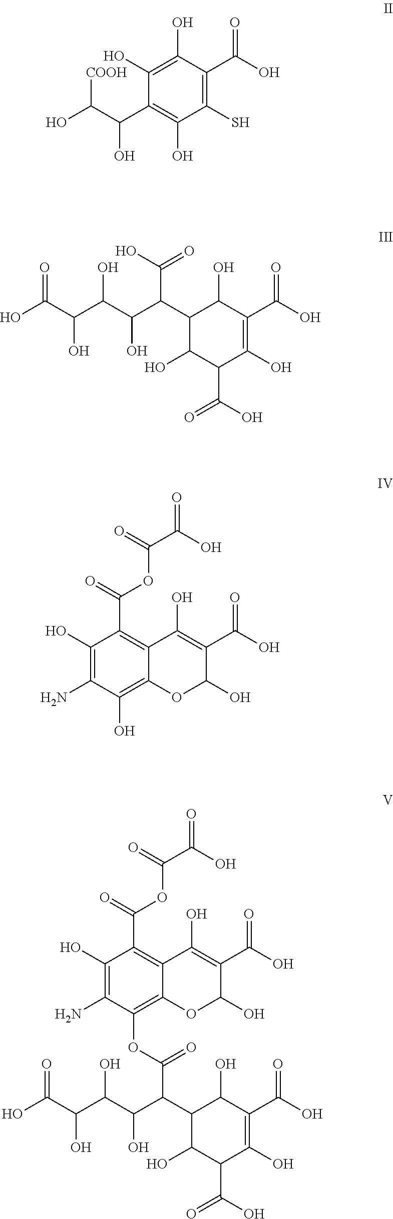 [SMILES: O=C(O)c1c(O)c(O)c(C(O)C(O)C(=O)O)c(O)c1S ; O=C(O)C1=C(O)C(C(=O)O)C(O)C(C(C(=O)O)C(O)C(O)C(O)C(=O)O)C1O ; Nc1c(O)c2c(c(C(=O)OC(=O)C(=O)O)c1O)C(O)=C(C(=O)O)C(O)O2 ; Nc1c(O)c(C(=O)OC(=O)C(=O)O)c2c(c1OC(=O)C(C(O)C(O)C(O)C(=O)O)C1C(O)C(C(=O)O)=C(O)C(C(=O)O)C1O)OC(O)C(C(=O)O)=C2O]

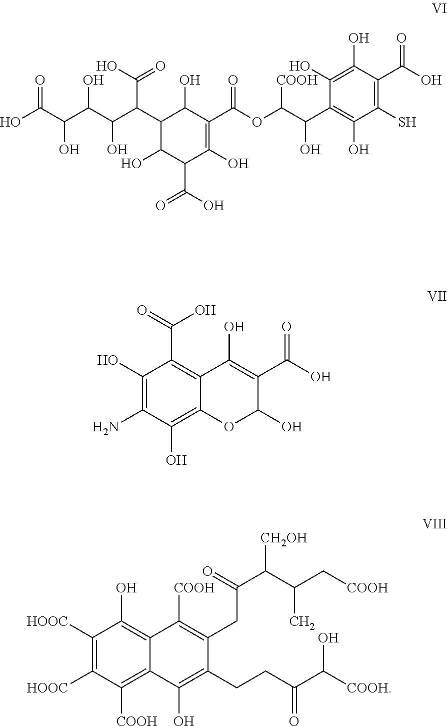 [SMILES: O=C(OC(C(=O)O)C(O)c1c(O)c(O)c(C(=O)O)c(S)c1O)C1=C(O)C(C(=O)O)C(O)C(C(C(=O)O)C(O)C(O)C(O)C(=O)O)C1O ; Nc1c(O)c2c(c(C(=O)O)c1O)C(O)=C(C(=O)O)C(O)O2 ; CC(CC(=O)O)C(CO)C(=O)Cc1c(CCC(=O)C(O)C(=O)O)c(O)c2c(C(=O)O)c(C(=O)O)c(C(=O)O)c(O)c2c1C(=O)O]

The anti-oxidation efficiency of these sustainable natural derivatives is due to their chemical structure which contains functional groups that are known as antioxidants and free radical scavenger active groups. Fulvic acid as a basic ingredient for the proposed technology is currently extracted on industrial scale mainly for use as organic fertilizers and plant nutrients. It is sourced cheaply from marsh and forest soils that can contain up to 30-40% humic materials. Furthermore, these compounds have tremendous potential as thermal stabilizers for various polymer systems used in food packaging.

Fulvic acid and derivatives of fulvic acid I have various uses including as antioxidants for several polymeric systems such as polyvinyl chloride (PVC), polyethylene (PE) and polypropylene (PP). These antioxidants can be used as a solid, solution, chelated with transition (nutrient) metals. The carboxylic acid group can be transformed into carboxylate salts of Na, K, Ca, Zn, Mg, and can form chelates with divalent and trivalent metal ions.

The antioxidants of embodiments from hydrolysis of fulvic acid require no necessary purification steps, such as ultrafiltration or desalination, nor fractionation into fractions with distinct molecular weights and high purity. The crude antioxidant hydrolysate solution advantageously, exhibits high activity without any further costly processing. However, purified compounds, their salts, chelates, and cleavage derivatives can potentially exhibit superior characteristics in certain applications.

Fulvic acid and fulvic acid hydrolysis products provide a stabilizing effect at various doses in foods. The antioxidant property in food of the crude extract, allows its usage at a low dose, hence, bypassing any undesired color or flavor effect that might arise from the use of the hydrolyzed fulvic acid as food preservatives.

Fulvic acid I cleavage derivatives derived from an alkaline hydrolysis technique, chemical compounds II-VII, all have several active functional groups (quinones, hydroquinones and alkyl phenols) that can act as anti-oxidants and free radical scavengers, chelating groups (via carboxyl groups adjacent to carbonyl or hydroxyl groups). It is also noted that the chemical structures detailed below are highly synchronized with that of ascorbic acid (Vitamin C), (see the sugar side group in structures III, V, and VI) the world's most popular and commonly used as food antioxidant. The chemical compounds I-VIII are less expensive to manufacture than ascorbic acid and have the ability to dissolve and bond minerals and other nutritional elements with enhanced bioavailability.

One embodiment is an antioxidant compound. An embodiment comprises at least one compound of formula II, formula III, formula IV, formula V, formula VI, formula VII, a salt, chelate, or combination thereof. A certain embodiment is an antioxidant of formula II, a salt, or chelate thereof. Another embodiment is an antioxidant of formula III, a salt, or chelate thereof. Still another embodiment is a compound of formula IV, a salt, or chelate thereof. Another certain embodiment is a compound of formula V, a salt, or chelate thereof. Another embodiment is a compound of formula VI, a salt, or chelate thereof. And yet another certain embodiment is a compound of formula VII, a salt, or chelate thereof.

In each of the aforementioned embodiments of the antioxidant compounds, the antioxidant compound may be a salt. In certain embodiments, the salt is a lithium salt, sodium salt, ammonium salt, potassium salt, calcium salt, barium salt, magnesium salt, manganese salt, zinc salt, aluminum salt, iron salt, or a combination thereof. In certain embodiments, the salt is a sodium salt, potassium salt, calcium salt, ammonium salt, or combination thereof. In an embodiment, the antioxidant compound is a sodium salt. In another embodiment, the antioxidant compound is a potassium salt. In yet another embodiment, the antioxidant compound is a calcium salt. In still another certain embodiment, the antioxidant compound is an ammonium ion salt. In yet other embodiments of the aforementioned antioxidant compounds, the antioxidant compound may be a transition metal ion chelate. In certain embodiments, the transition metal ion chelate is a manganese salt, zinc salt, aluminum salt, iron salt, or a combination thereof.

Another embodiment is a method of preparing an antioxidant compound, the method comprising substantially hydrolyzing fulvic acid I to form at least one antioxidant compound of formula II, formula III, formula IV, formula V, formula VI, formula VII, a salt, or chelate thereof. In a further embodiment, the fulvic acid comprises at least one compound of formula I.

In various embodiments, the hydrolysis is performed at about reflux temperature. In any of the above embodiments, the hydrolyzing is carried out using an alkaline solution. The alkaline solution may be a pH of at or about 8, 8.5, 9, 9.5, 10, 11, 12, 13, or 14, or within a range between any two values. In a certain embodiment, the alkaline solution may be a pH of 8.5-14. The alkaline solution may contain lithium hydroxide, sodium hydroxide, potassium hydroxide, or combinations thereof.

In each of the aforementioned embodiments of the methods of preparation, the method may further comprise neutralizing the hydrolyzing solution. The solution may be neutralized to a pH of about 8.5, 8, 7.5, 7, 6.5, 6, 5, 4, 3, 2, or 1, or within a range between any two values. In a certain embodiment, the hydrolyzing solution is neutralized to a pH of about 5 to about 8.5. In some embodiments, the neutralization is carried to an acidic pH.

Some embodiments include forming at least one compound of formula II, formula III, formula IV, formula V, formula VI, and formula VII as a salt. In certain embodiments, the salt is a lithium salt, sodium salt, ammonium salt, potassium salt, calcium salt, barium salt, magnesium salt, manganese salt, zinc salt, aluminum salt, iron salt, or a combination thereof.

In an embodiment, the fulvic acid I is hydrolyzed at reflux for about 15 minutes, about 30 minutes, about 45 minutes, about one hour, about 1.5 hours, or a period of time between or including any two of the time periods, wherein the hydrolysis product comprises greater percentages of compounds V and VI than compounds II, III, VI, VII and vanillin. In other embodiments, the fulvic acid I is hydrolyzed for periods of time of about 2 hours, about 4 hours, about 8 hours, about 24 hours, or a period of time between or including any two of the time periods, wherein the hydrolysis product comprises greater percentages of compounds II, III, VI, VII and vanillin than compounds V and VI. In various embodiments, the reaction rates are adjusted for the Arrheneous equation, k=A exp(−Eₐ/RT). In certain embodiments, the recited reaction times are reduced by half for each about 10° C. increase in temperature. In other certain embodiments, the recited reaction times are doubled for each about 10° C. decrease in temperature.

In each of the aforementioned embodiments of the methods of preparation, the method may further comprise isolating at least one of the antioxidant compounds. In other embodiments the isolating prepares a mixture of more than one of the compounds of formula II, formula III, formula IV, formula V, formula VI, formula VII, a salt, or chelate thereof. In certain embodiments, the salt isolated is a lithium salt, sodium salt, ammonium salt, potassium salt, calcium salt, barium salt, magnesium salt, manganese salt, zinc salt, aluminum salt, iron salt, or a combination thereof.

Another embodiment is a stabilized food product comprising at least one antioxidant compound of formula I-VIII, a salt, or chelate thereof in a food product matrix. In certain embodiments, the stabilized food product comprises at least one antioxidant compound of formula II-V, a salt or chelate thereof in a food product matrix. In other embodiments, the stabilized food product comprises at least one antioxidant compound of formula I or VIII, a salt or chelate thereof in a food product matrix. In a certain embodiment, the food product matrix is a dairy product, fat, oil, fat emulsion, edible ice, fruit, vegetable, fungi, seaweed, nuts, seeds, confectionery, cereal, cereal product derived from cereal grains, bakery ware, meat, meat byproduct, fish, fish product, egg product, sugar, artificial sweetener, spices, condiment, soup, sauce, salad, protein mix, non-dairy beverage, savory snack, or combinations thereof. In another certain embodiment, the food product matrix is a vegetable oil, animal fat, processed cheeses, chewing gum base, processed meat products, dried meats, sausages, beef patties, meatballs, frozen seafood, pizza toppings, protein, yeast, bakery products, dry cereals, spices, dehydrated potatoes, potato chips, beverage mixes, nonalcoholic beverages, mixed nuts, fruit, vegetables, butter, margarine, dairy products, or combinations thereof.

Yet another embodiment is a stabilized food product comprising at least one antioxidant compound of formula II-VII, a salt, or chelate thereof in a food product matrix, wherein the antioxidant compound extends the shelf-life of the food product matrix. In another embodiment is a stabilized food product comprising at least one antioxidant compound of formula I-VIII, a salt, or chelate thereof in a food product matrix, wherein the antioxidant compound extends the shelf-life of the food product matrix. In still another embodiment, the stabilized food product is stable to about 250-350° C. according to the stabilizing composition. In yet another embodiment, the antioxidant compound is present in the food product matrix or other stabilized matrices at a concentration of about 1 percent, about 3000 ppm, about 1000 ppm, about 300 ppm, about 100 ppm, about 30 ppm, about 10 ppm, about 3 ppm, about 1 ppm, or any range between two of the concentrations. In various specific embodiments, the antioxidant is present in a food product at less than about 3000 ppm, less than about 300 ppm, less than about 100 ppm, or less than about 10 ppm.

Another embodiment is a polymeric matrix comprising at least one antioxidant compound of formula II-VII a salt, chelate, or combination thereof, in combination with a polymer. In still another embodiment is a polymeric matrix comprising at least one antioxidant compound of formula I-VIII a salt, chelate, or combination thereof, in combination with a polymer. In some embodiments, the antioxidant compound is an antioxidant. In certain embodiments, the polymer is a polyvinyl chloride, low density polyethylene, high density polyethylene, polyvinyl alcohol, polypropylene, or combination thereof. In yet other embodiments, the polymeric matrix is a food packaging.

In yet another embodiment, the antioxidant compound is present in the polymer matrices by weight at a concentration of about 5%, about 2.5%, about 1%, about 0.5%, about 1000 ppm, about 300 ppm, about 200 ppm, about 100 ppm, about 30 ppm, about 10 ppm, about 3 ppm, about 1 ppm, or any range between two of the concentrations. In various specific embodiments, the antioxidant is present in a polymer at less than about 5000 ppm, less than about 300 ppm, less than about 100 ppm, or less than about 10 ppm. In certain embodiments, the antioxidant compound is between about 0.02% and about 2.5% by weight of the polymer. In another certain embodiment, the antioxidant compound is between about 0.1% and about 0.5% by weight.

In each of the aforementioned embodiments of the polymer matrix, the antioxidant compound may be a salt. In certain embodiments, the salt is a lithium salt, sodium salt, ammonium salt, potassium salt, calcium salt, barium salt, magnesium salt, manganese salt, zinc salt, aluminum salt, iron salt, or a combination thereof. In certain embodiments, the salt is a sodium salt, potassium salt, calcium salt, ammonium salt, or combination thereof. In an embodiment, the antioxidant compound is a sodium salt. In another embodiment, the antioxidant compound is a potassium salt. In yet another embodiment, the antioxidant compound is a calcium salt. In still another certain embodiment, the antioxidant compound is an ammonium ion salt. In yet other embodiments of the aforementioned antioxidant compounds, the antioxidant compound may be a transition metal ion chelate. In certain embodiments, the transition metal ion chelate is a manganese salt, zinc salt, aluminum salt, iron salt, or a combination thereof.

Another embodiment is a nutritional supplement comprising at least one antioxidant compound of formula II-VII a salt, chelate, or combination thereof, in combination with a nutritionally acceptable carrier. Yet another embodiment is a nutritional supplement comprising at least one antioxidant compound of formula I-VIII, a salt, chelate, or combination thereof, in combination with a nutritionally acceptable carrier. In some embodiments, the nutritionally acceptable carrier further comprises a flavoring agent. In other embodiments, the nutritional supplement further comprises buffering agents. In still other embodiments, the nutritional supplement further comprises minerals. Yet other embodiments are wherein the antioxidant compound is a salt, chelate, or combination thereof. Further, in some embodiments, the salt is a lithium salt, sodium salt, ammonium salt, potassium salt, calcium salt, barium salt, magnesium salt, manganese salt, zinc salt, aluminum salt, iron salt, or a combination thereof. By nutritional supplements we include as examples, but are not limited to: eye health supplements, vitamins, nutrition boosters, carotenoid supplements, protein supplements, energy bars, nutritional bars, algal oils, fish oils, and oils containing polyunsaturated fatty acids.

Yet another embodiment is a method of preparing a stabilized food product comprising adding at least one antioxidant compound of formula II-VII, a salt, or chelate thereof to a food product matrix. In some embodiments, the antioxidant compound added to the food product matrix is a compound of formula II-V, a salt, or chelate thereof. In other embodiments, the antioxidant compound added to the food product matrix is at least one compound of formula I-VIII, a salt, or chelate thereof. In certain embodiments, the compound is a salt, chelate, or combination thereof. In some embodiments, the food product matrix is a dairy product, fat, oil, fat emulsion, edible ice, fruit, vegetable, fungi, seaweed, nuts, seeds, confectionery, cereal, cereal product derived from cereal grains, bakery ware, meat, meat byproduct, fish, fish product, egg product, sugar, artificial sweetener, spices, condiment, soup, sauce, salad, protein mix, non-dairy beverage, savory snack, or combinations thereof. In other embodiments, the food product matrix is a vegetable oil, animal fat, processed cheeses, chewing gum base, processed meat products, dried meats, sausages, beef patties, meatballs, frozen seafood, pizza toppings, protein, yeast, bakery products, dry cereals, spices, dehydrated potatoes, potato chips, beverage mixes, nonalcoholic beverages, mixed nuts, fruit, vegetables, butter, margarine, dairy products, or combinations thereof.

The instant fulvic acid and fulvic acid hydrolysate compounds may be added to foods, including animal foods. By foods we mean both human and animal foods. By human foods we include as examples, but are not limited to: meat (wild and domestic; fresh and cured, processed and unprocessed, dried, canned), Poultry, fish, vegetable protein, dairy products (milk, cheese, yogurt, ice cream), ground spices, vegetables, pickles, mayonnaise, sauces (pasta sauces, tomato based sauces), salad dressings, dried fruits, nuts, potato flakes, soups, baked goods (breads, pastries, pie crusts, rolls, cookies, crackers, cakes, pies, bagels), vegetable oils, frying oil, fried foods (potato chips, corn chips), prepared cereals (breakfast cereals), cereal grain meals, condiments (ketchup, mustard, cocktail sauce, candies, confectionary, chocolates, baby foods). By animal foods we include as examples, but are not limited to: extruded pet food, kibbles, dry pet food, semi-dry pet food, and wet pet food.

The instant fulvic acid and fulvic acid hydrolysate compounds may be added directly to foods according to the solubility characteristics. They may be dissolved in a carrier, such as water, ethanol, glycerin, food grade surfactants, and the like, and then added to foods. They can be dispersed onto solid carriers, such as salt, flour, sugars, maltodextrin, silica (such as CABOSIL™), cyclodextrins, starches, gelatins, lactose, whey powders, proteins, and the like and then added to foods.

Yet another embodiment is a method for preparing a wherein the antioxidant extends the shelf-life of the food product matrix. In still another embodiment, the method produces a stabilized food product that is stable to about 350° C. In yet another embodiment, the method comprises adding the antioxidant compound to the food product matrix at a concentration of about 1 percent, about 3000 ppm, about 1000 ppm, about 300 ppm, about 100 ppm, about 30 ppm, about 10 ppm, about 3 ppm, about 1 ppm, or any range between two of the concentrations. In various embodiments, the antioxidant is present in a food product at less than about 3000 ppm, less than about 300 ppm, less than about 100 ppm, or less than about 10 ppm.

The instant fulvic acid and fulvic acid hydrolysate compounds may be added to cosmetics. By cosmetics we include as examples, but are not limited to: lip balm, lip gloss, lipstick, lip stains, lip tint, blush, bronzers & highlighters, concealers & neutralizers, foundations, foundation primer, glimmers & shimmers, powders, eye shadow, eye color, eye liner, mascara, nail polish, nail treatments-strengtheners, make-up, body creams, moisturizers, suntan preparations, sunless tan formulations, body butter, body scrubs, make-up remover, shampoos, conditioners, dandruff control formulations, anti-frizz formulations, straightening formulations, volumizing formulations, styling aids, hairsprays, hair gels, hair colors and tinting formulations, anti-aging creams, body gels, essential oils, creams, cleansers, soaps.

A further embodiment introduces a mechanism to retard complex formation following preparation of the fulvic acid derivative's carboxylic groups to salts of sodium, calcium, etc. which would free other antioxidant active groups to be effective food preservative anti-oxidants. This is similar to using calcium or sodium salt of ascorbic acid as antioxidants instead of ascorbic acid.

Consumer interest in and awareness of the health properties of antioxidants has been increasing in recent years. This has simultaneously increased global sales of antioxidants (whether used as a food preservative or to provide a health enhancing or functional benefit) and foods that are recognized as being naturally rich in antioxidants. Notable examples include certain varieties of fruit such as blueberries and blackberries. As the sector has developed, antioxidants are now being used in the manufacture of a greater variety of foods to cater for increasingly health-conscious consumers. This has been most apparent in sectors such as chocolate confectionery, soft drinks, and hot beverages such as tea.

The fulvic acid and fulvic acid derivatives are more efficient (based on fulvic acid studies) and cost effective compared to all other food antioxidants. Fulvic acid and its derivatives would be cheaper to manufacture than ascorbic acid and synthetic antioxidants and have greater thermal stability convenient for almost all food processing and cooking up to 350° C. and have the ability to dissolve and bond minerals and other nutritional elements with enhanced bioavailability.

### EXAMPLES

Although the present technology has been described in considerable detail with reference to certain preferred embodiments thereof, other versions are possible. Therefore the spirit and scope of the appended claims should not be limited to the description and the preferred versions contained within this specification. Various aspects of the present technology will be illustrated with reference to the following non-limiting examples.

### Example 1

### Preparation of Antioxidant Compounds from Fulvic Acid

Preparation of antioxidant compounds from fulvic acid I. Fulvic acid was extracted from marsh soils having an organic content of 30-35%. The extraction was carried out by treating the soil with 0.5 mole sodium hydroxide per 100 g. The solution was neutralized by 5% HCl to a pH of about 2. The precipitated humic acid was separated. The portion that was soluble in acidic and neutral solution (Fulvic acid) was transferred to alkaline solution by adding 20% sodium hydroxide solution at a pH of 12-14. The fulvic acid alkaline solution containing 100 g of fulvic acid was heated at 110° C. and 1.5 atmosphere in laboratory scale autoclave. Fractions of the hydrolysis solution were taken every 30 minutes from the first hour to the fourth hour, whereupon the homogeneous reaction mixture was neutralized with acid, then transferred to the carboxylate salts by using calcium hydroxide to give the calcium fulvate salt. Other carboxylate salts (Ba, Mg, Mn, etc.) may be made by neutralizing with the corresponding alkaline hydroxide. The carboxyl equivalent was determined by IR, titration, and thermal analysis.

### Example 2

### Fulvic Acid Derived Antioxidants and Thermal Stabilizers in Polymers

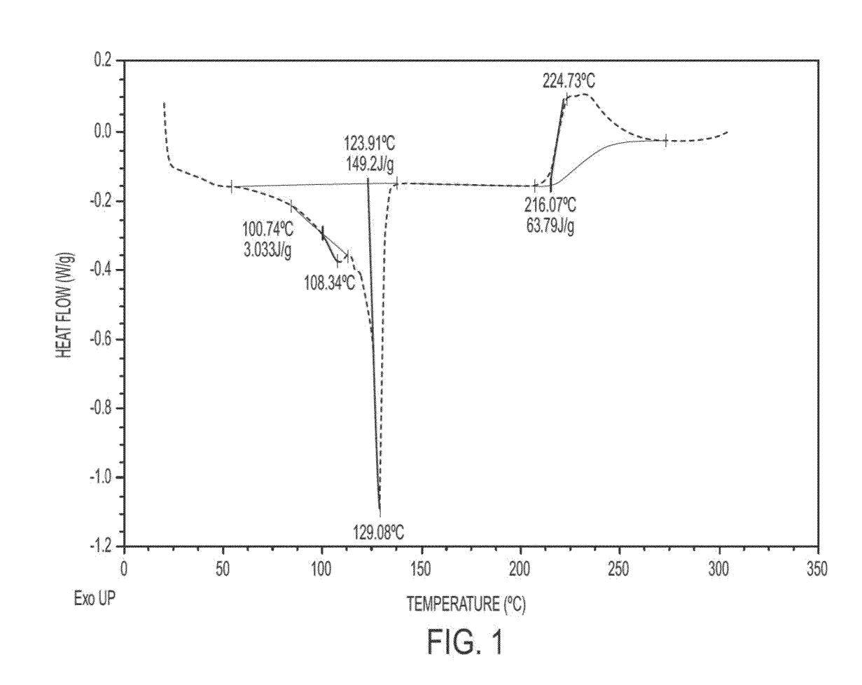

The degradation products of Example 1, recovered after three hours of hydrolysis, were evaluated as antioxidants and thermal stabilizers for PVC, low density polyethylene (LDPE), and polyvinyl alcohol (PVA) to evaluate the anti-oxidation efficiency using thermal analysis techniques (DSC, TGA, and DTG). The thermooxidative enthalpy change from DSC curves (e.g. FIGS. 1-3) and the % of HCl loss as a function of temperature represent the stability of polyethylene and PVC respectively. Typical obtained analysis data are shown in Table (1). The data shows PVC with 1% of fulvic acid and PVC with 1% of calcium fulvate hydrolysis mixture were more stable than the PVC control polymer.

TABLE (1)
Thermal degradation stability of PVC and stabilized PVC

| | HCl % (loss) from TGA | | |
|---|---|---|---|
| | | | PVC with 1% |
| | | PVC with | Ca-Fulvate |
| | PVC control | 1% Fulvic | hydrolysed |
| Temperature (° C.) | (K value 70) | acid | mixture |
| 250 | 3 | 1 | 0 |
| 260 | 5 | 2 | 0 |
| 270 | 10 | 3 | 0.5 |
| 280 | 33 | 5 | 1.0 |
| 290 | 39 | 14 | 1.0 |
| 300 | 47 | 33 | 5.0 |
| Total HCl loss (%) at | 63 | 57 | 53 |
| Decomp. Temp. | | | |
| Decomp. Temp., ° C. from | 265 | 300 | 310 |
| DTG | | | |
| Rate of decomposition at | 1.63 | 0.73 | 0.73 |
| Decomp. Temp. %/min | | | |

Typical DSC thermograms are shown in FIGS. 1-3 for polyethylene stabilized with 0.1% and 0.5% of calcium salts of antioxidants produced by hydrolysis of fulvic acid for three hours as in Example 1. The oxidation energy decreased from 63.79 J/g to 51.33 J/g when stabilized with 0.1%, and 23.97 J/g for the stabilized sample with 0.5% fulvic acid 3 hrs hydrolyzed product calcium salts.

FIG. 1 illustrates the thermo-oxidation differential scanning calorimetry (DSC) thermogram for un-stabilized LDPE. The first endothermic peak at 129.08° C. is the melting peak of the polymer while the second peak at 224.73° C. represents the exothermic thermo oxidation enthalpy change (63.79 J/g) that took place during this heating sequence in the presence of air.

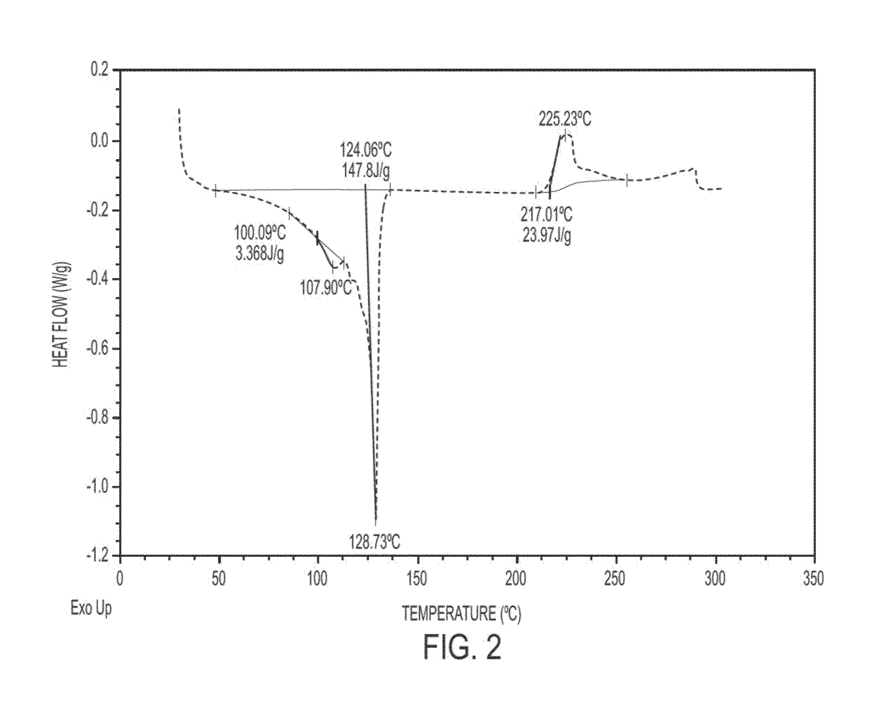

FIG. 2 is the thermo-oxidation DSC thermogram for low density polyethylene (LDPE) stabilized with 0.1 w/w % of the calcium salts prepared from fulvic acid by alkaline hydrolysis for three hours. The first endothermic peak at 125.77° C. is the melting peak of the polymer while the second peak at 219.69° C. represents the exothermic thermo oxidation (51.33 J/g) that took place during this heating sequence in the presence of air. The exothermic oxidation is reduced compared to the oxidation energy evolved from the non-stabilized sample.

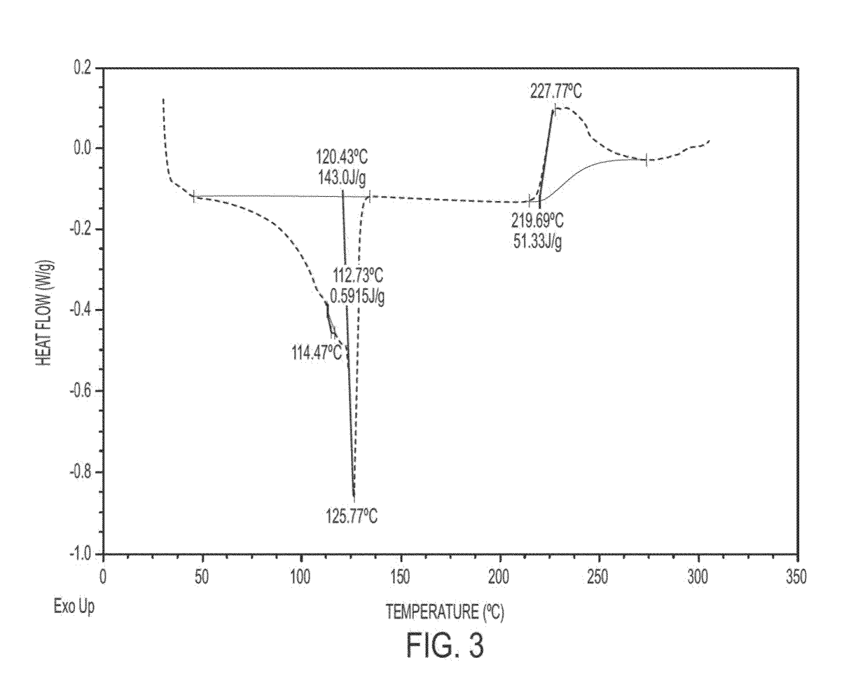

FIG. 3 is the thermo-oxidation DSC thermogram for LDPE stabilized with 0.5 w/w % of the calcium salts prepared from fulvic acid by alkaline hydrolysis for three hours. The first endothermic peak at 128.73° C. is the melting peak of the polymer while the second peak at 225.23° C. represents the exothermic oxidation (23.97 J/g) that took place during this heating sequence in the presence of air (FIG. 3). The exothermic oxidation is less than half of the oxidation energy evolved from the non-stabilized sample.

Photo oxidative degradation forms free radicals by bond breaking. The free radicals then react with oxygen to produce peroxy radicals which leads to further degradation via a well-established chain reaction mechanism. Oxidation of polyethylene tends to occur at weak links in the chain, such as branch points in low density polyethylene. The lower oxidation energy determined by DSC indicates the efficiency of these new antioxidants.

### Example 3

### Salts of Antioxidant Compounds for Stabilization of Polyvinylchloride

The carboxylate salts of the hydrolyzed (3 hour fraction) product as Ca, Mg, Ba, Zn, Fe, and their chelates with these metal ions showed remarkable efficiency for thermo-oxidative stabilization of polyvinylchloride (PVC) see Table (1). The mechanism of stabilizing PVC is based on capturing the evolved HCl from dehydrochlorination by the metal ions and the transition metal, accordingly deactivating the catalytic role of HCl in the thermal decomposition.

### Example 4

### Fulvic Acid Derived Antioxidant Compounds as Stabilizers for Canola Oil

Stabilized canola oil is compared to unstabilized canola oil. Unfortified canola oil, 100 g, is thoroughly mixed with 400 mL of deionized water and 10 g of polysorbate-20 (Tween 20). The blended emulsion is homogenized and then stored at 60° C. Emulsion solutions containing various concentrations (50 ppm, 100 ppm, 200 ppm) of antioxidant extracts of Example 1 are prepared and incubated at 60° C. on an orbital shaker along with a control treatment without antioxidants. Measurements are taken once a day, for several consecutive days by measuring the UV absorbance of the conjugated dienes of the canola oil at 234 nm. The absorbance (at A=234 nm) plotted against time shows the canola oil with antioxidants is more stable than the control without antioxidants.

### Example 5

### Fulvic Acid Derived Antioxidant Compounds as Stabilizers for Margarine

Margarine with added fulvate derived antioxidant compounds is more stable in comparison to unstabilized margarine. Margarine samples are prepared, containing: the antioxidant compounds of Example 1 (200 ppm), and no antioxidants (control), and incubated at 22-23° C., in the dark. Margarine samples of each treatment are pulled periodically, and the fat is separated by melting at 60° C., followed by centrifuging at about 1,000 g and decanting the upper (fat) phase. Oxidation is evaluated by measuring the peroxide value according to the AOCS official method Cd 8b-90. Results show that the instant antioxidant compounds inhibit oxidation in comparison to the untreated control, reflecting in lower levels of peroxide value (PV) over time.

### Example 6

### Fulvic Acid Derived Antioxidant Compounds as Stabilizers for Milk

Milk with added fulvate derived antioxidant compounds is more stable in comparison to unstabilized milk Fresh milk is treated with the antioxidant compounds of Example 1 at 100 ppm, homogenized, spray-dried, then incubated at room temperature (22-23° C.), in the dark, in comparison to the same spray-dried milk without any antioxidant compounds added (control). Samples are analyzed periodically by gas chromatography, and oxidation is traced by monitoring the generation and accumulation of secondary oxidation products (e.g. hexanal). The experiment shows an antioxidative protective effect of the instant antioxidant compounds, reflected in lower levels of secondary oxidation products.

### Example 7

### Fulvic Acid Derived Antioxidant Compounds as Stabilizers for Breakfast Cereal

Breakfast cereal with added fulvate derived antioxidant compounds is more stable in comparison to unstabilized breakfast cereal. Breakfast cereal consisting of 5% milled flax seed and 95% corn semolina, and 250 ppm of the antioxidant compounds of Example 1, is compared to the same recipe without any antioxidant additives (control). Samples are incubated in the dark at room temperature (22-23° C.) for several weeks. The cereal sample containing the instant antioxidant compounds is more oxidatively stable as it exhibits lower levels of the oxidation markers as detected by gas chromatography.

### Example 8

### Fulvic Acid Derived Antioxidant Compounds as Stabilizers for Food Packaging

The oxidation stability was evaluated for the different constituents of the fulvic acid, its carboxylate salts of Ca, Mg, Ba, Zn, Fe and its hydrolysis product as prepared in Example 1 using the food products of Examples 4-7. The fulvic acid hydrolysis products showed higher antioxidant efficiency than fulvic acid itself.

### Example 9

### Stabilized Polyethylene for Food Packaging

Polyethylene, stabilized with 0.1%, 0.5% of fulvic acid, fulvic acid derivatives, salts, or their chelates are extruded into films for film packaging (typical DSC curves shown in FIGS. 1-3). Films are subjected to the natural sun light in Sydney, Australia, with high UV light for three weeks. The films are then tested by IF and UV spectrometry. The stabilized films with metal chelates show no color change and spectral changes, while control, unstabilized polyethylene samples show change in the absorption spectra and coloration.

### Example 10

### Stabilized Polyvinyl Chloride for Food Packaging

Polyvinyl chloride, K value 70, stabilized with 1% fulvic acid, fulvic acid derivatives, salts, or their chelates are extruded into films for film packaging (typical thermal stability characteristics listed in Table 1) are extruded to films for food packaging, films are subjected to natural sun light in Sydney, Australia, with high UV light for 3 weeks. The films stabilized with metal chelates show no color change while control, unstabilized PVC samples show change to a dark yellowish color.

1. An antioxidant compound comprising at least one compound of formula II, formula III, formula IV, formula V, formula VI, formula VII, formula VIII, a salt, chelate, or combination thereof:

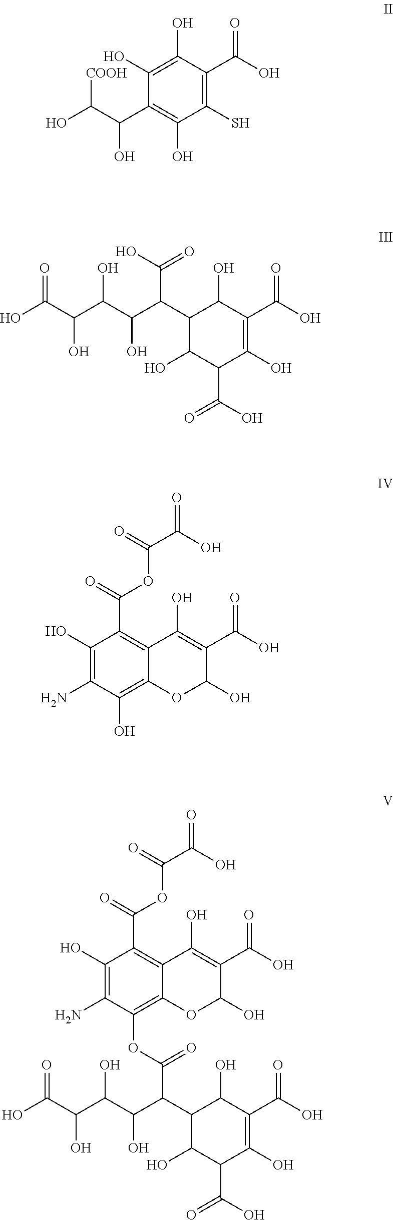 [SMILES: O=C(O)c1c(O)c(O)c(C(O)C(O)C(=O)O)c(O)c1S ; O=C(O)C1=C(O)C(C(=O)O)C(O)C(C(C(=O)O)C(O)C(O)C(O)C(=O)O)C1O ; Nc1c(O)c2c(c(C(=O)OC(=O)C(=O)O)c1O)C(O)=C(C(=O)O)C(O)O2 ; Nc1c(O)c(C(=O)OC(=O)C(=O)O)c2c(c1OC(=O)C(C(O)C(O)C(O)C(=O)O)C1C(O)C(C(=O)O)=C(O)C(C(=O)O)C1O)OC(O)C(C(=O)O)=C2O]

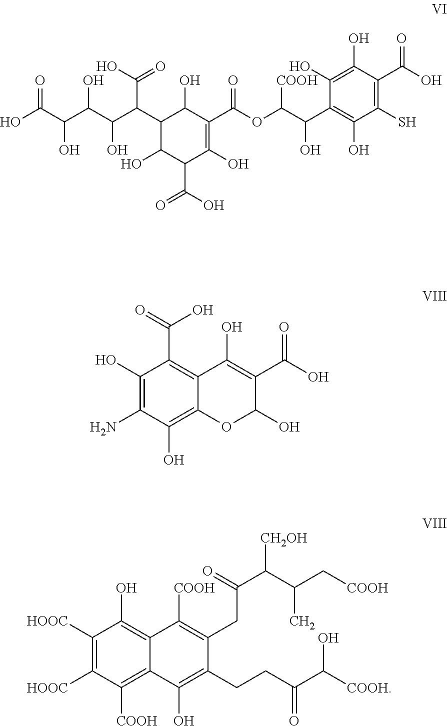 [SMILES: O=C(OC(C(=O)O)C(O)c1c(O)c(O)c(C(=O)O)c(S)c1O)C1=C(O)C(C(=O)O)C(O)C(C(C(=O)O)C(O)C(O)C(O)C(=O)O)C1O ; Nc1c(O)c2c(c(C(=O)O)c1O)C(O)=C(C(=O)O)C(O)O2 ; CC(CC(=O)O)C(CO)C(=O)Cc1c(CCC(=O)C(O)C(=O)O)c(O)c2c(C(=O)O)c(C(=O)O)c(C(=O)O)c(O)c2c1C(=O)O]

2. The antioxidant compound of claim 1, wherein at least one compound is formula II-VII, a salt, or chelate thereof.

3. The antioxidant compound of claim 1, wherein the compound is formula II, a salt, or chelate thereof.

4. The antioxidant compound of claim 1, wherein the compound is formula III, a salt, or chelate thereof.

5. The antioxidant compound of claim 1, wherein the compound is formula IV, a salt, or chelate thereof.

6. The antioxidant compound of claim 1, wherein the compound is formula V, a salt, or chelate thereof.

7. The antioxidant compound of claim 1, wherein the compound is formula VI, a salt, or chelate thereof.

8. The antioxidant compound of claim 1, wherein the compound is formula VII, a salt, or chelate thereof.

9. The antioxidant compound of claim 1, wherein the compound is formula VIII, a salt, or chelate thereof.

10. The antioxidant compound of claim 1, wherein the salt is selected from the group consisting of a lithium salt, a sodium salt, an ammonium salt, a potassium salt, a calcium salt, a barium salt, a magnesium salt, a manganese salt, a zinc salt, an aluminum salt, and an iron salt.

11. The antioxidant compound of claim 1, wherein the salt comprises sodium salt, potassium salt, calcium salt, or ammonium salt.

12. The antioxidant compound of claim 1, wherein the chelate comprises a transition metal ion chelate.

13. A method of preparing an antioxidant compound, the method comprising:
hydrolyzing a fulvic acid to form at least one antioxidant compound of formulas II-VII, a salt, or chelate thereof:

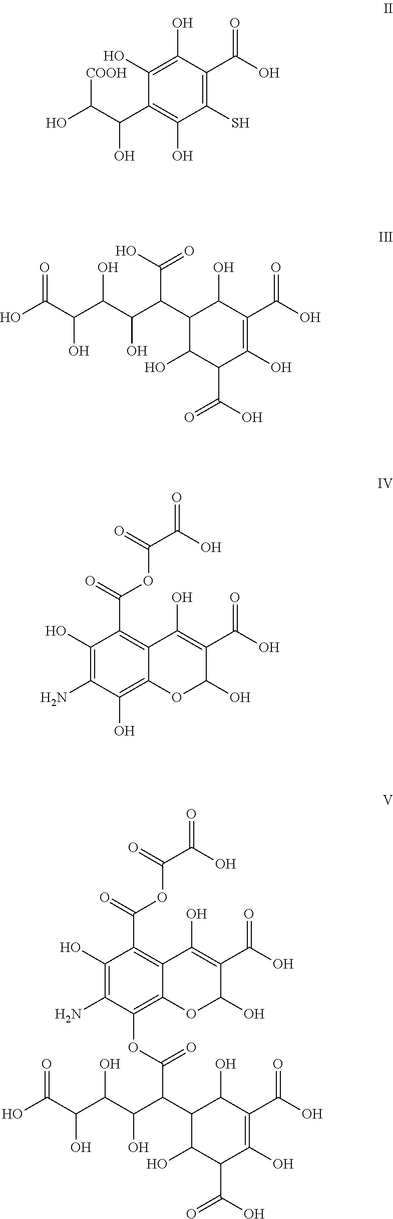 [SMILES: O=C(O)c1c(O)c(O)c(C(O)C(O)C(=O)O)c(O)c1S ; O=C(O)C1=C(O)C(C(=O)O)C(O)C(C(C(=O)O)C(O)C(O)C(O)C(=O)O)C1O ; Nc1c(O)c2c(c(C(=O)OC(=O)C(=O)O)c1O)C(O)=C(C(=O)O)C(O)O2 ; Nc1c(O)c(C(=O)OC(=O)C(=O)O)c2c(c1OC(=O)C(C(O)C(O)C(O)C(=O)O)C1C(O)C(C(=O)O)=C(O)C(C(=O)O)C1O)OC(O)C(C(=O)O)=C2O]

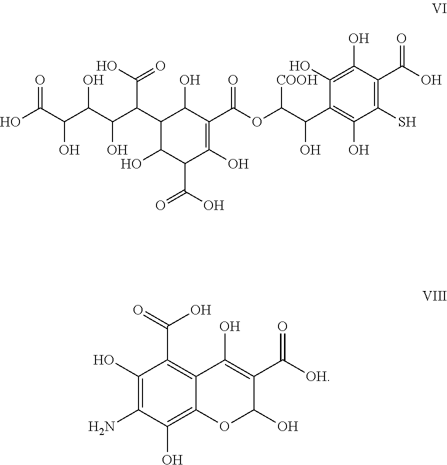 [SMILES: O=C(OC(C(=O)O)C(O)c1c(O)c(O)c(C(=O)O)c(S)c1O)C1=C(O)C(C(=O)O)C(O)C(C(C(=O)O)C(O)C(O)C(O)C(=O)O)C1O ; Nc1c(O)c2c(c(C(=O)O)c1O)C(O)=C(C(=O)O)C(O)O2]

14. The method of claim 13, wherein hydrolyzing the fulvic acid comprises hydrolyzing at least one compound of formula I: 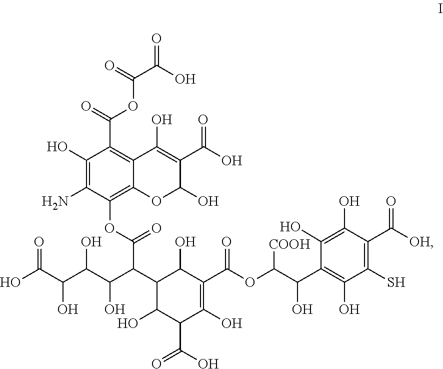 [SMILES: Nc1c(O)c(C(=O)OC(=O)C(=O)O)c2c(c1OC(=O)C(C(O)C(O)C(O)C(=O)O)C1C(O)C(C(=O)OC(C(=O)O)C(O)c3c(O)c(O)c(C(=O)O)c(S)c3O)=C(O)C(C(=O)O)C1O)OC(O)C(C(=O)O)=C2O]
a salt, or chelate thereof.

15. The method as in claim 13, wherein the hydrolyzing comprises hydrolyzing at about the reflux temperature of the fulvic acid.

16. The method as in claim 13, wherein the hydrolyzing comprises hydrolyzing under at least 1.3 bar pressure.

17. The method as in claim 13, wherein the hydrolyzing comprises hydrolyzing using an alkaline solution at a pH of about 8.5 to about 14.

18. (canceled)

19. The method as in claim 17, wherein the alkaline solution comprises an aqueous lithium hydroxide, an aqueous sodium hydroxide, an aqueous potassium hydroxide, or combination thereof.

20. The method as in claim 13, further comprising neutralizing the at least one antioxidant compound to a pH of about 5 to about 8.5.

21. (canceled)

22. The method as in claim 13, further comprising acidifying the at least one antioxidant compound to an acidic pH.

23. The method as in claim 13, wherein hydrolyzing comprises forming a salt of the at least one compound of formula II-VII.

24. The method as in claim 23, wherein the salt is a lithium salt, a sodium salt, an ammonium salt, a potassium salt, a calcium salt, a barium salt, a magnesium salt, a manganese salt, a zinc salt, an aluminum salt, an iron salt, or a combination thereof.

25. The method as in claim 13, further comprising isolating the at least one antioxidant compound of formulas II-VII.

26. The method as in claim 13, wherein the hydrolyzing comprises hydrolyzing to a temperature above 25° C. to about reflux temperature of the fulvic acid, and isolating the at least one antioxidant compound of formula II, III, V, VI, VII or combinations thereof.

27. The method as in claim 13, wherein the hydrolyzing comprises hydrolyzing for lesser than 60 minutes and at a temperature of at least about 45° C.

28.-29. (canceled)

30. The method as in claim 13, further comprising isolating at least one antioxidant compound of formula II, III, VI, VII, or combinations thereof.

31. A stabilized food product comprising at least one antioxidant compound of formula I-VIII, a salt, or chelate thereof: 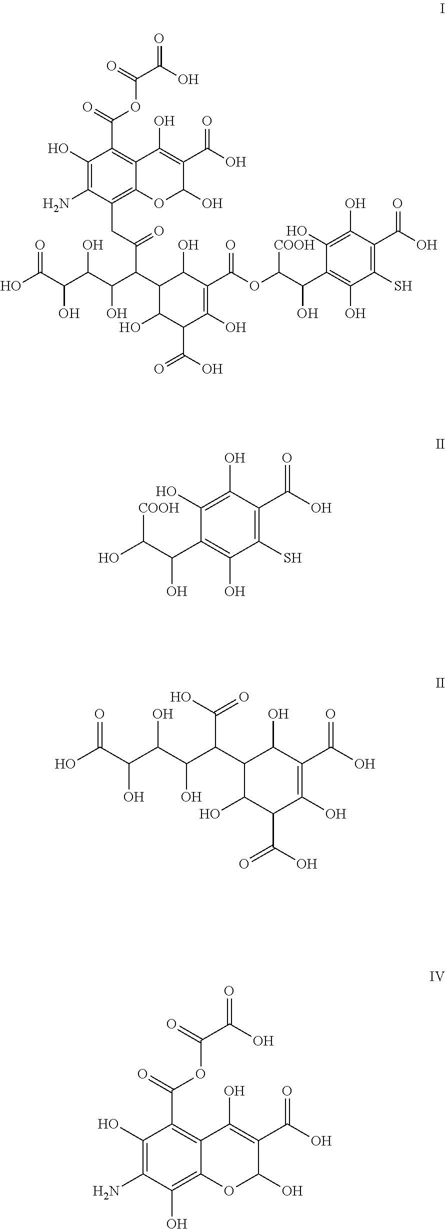 [SMILES: Nc1c(O)c(C(=O)OC(=O)C(=O)O)c2c(c1CC(=O)C(C(O)C(O)C(O)C(=O)O)C1C(O)C(C(=O)OC(C(=O)O)C(O)c3c(O)c(O)c(C(=O)O)c(S)c3O)=C(O)C(C(=O)O)C1O)OC(O)C(C(=O)O)=C2O ; O=C(O)c1c(O)c(O)c(C(O)C(O)C(=O)O)c(O)c1S ; O=C(O)C1=C(O)C(C(=O)O)C(O)C(C(C(=O)O)C(O)C(O)C(O)C(=O)O)C1O ; Nc1c(O)c2c(c(C(=O)OC(=O)C(=O)O)c1O)C(O)=C(C(=O)O)C(O)O2] 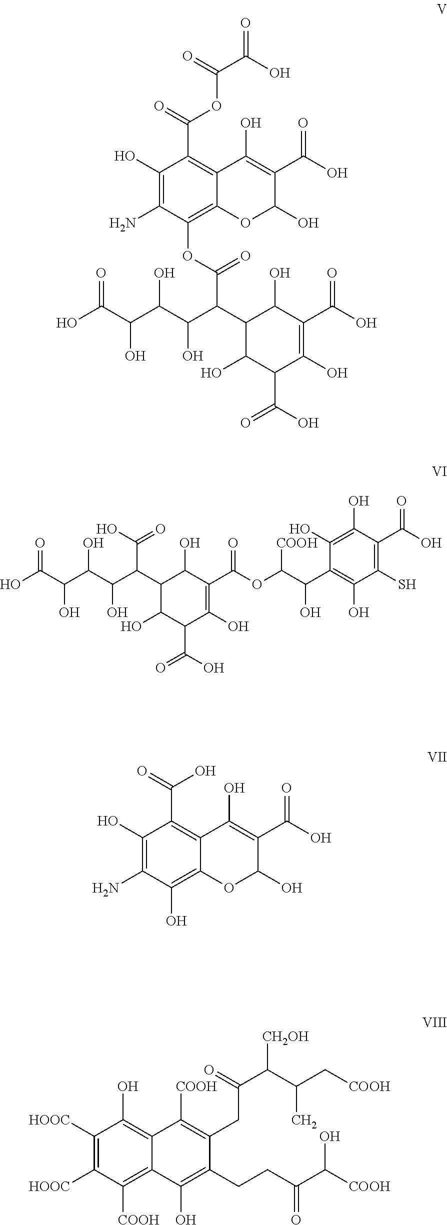 [SMILES: Nc1c(O)c(C(=O)OC(=O)C(=O)O)c2c(c1OC(=O)C(C(O)C(O)C(O)C(=O)O)C1C(O)C(C(=O)O)=C(O)C(C(=O)O)C1O)OC(O)C(C(=O)O)=C2O ; O=C(OC(C(=O)O)C(O)c1c(O)c(O)c(C(=O)O)c(S)c1O)C1=C(O)C(C(=O)O)C(O)C(C(C(=O)O)C(O)C(O)C(O)C(=O)O)C1O ; Nc1c(O)c2c(c(C(=O)O)c1O)C(O)=C(C(=O)O)C(O)O2 ; CC(CC(=O)O)C(CO)C(=O)Cc1c(CCC(=O)C(O)C(=O)O)c(O)c2c(C(=O)O)c(C(=O)O)c(C(=O)O)c(O)c2c1C(=O)O]
in a food product matrix.

32.-34. (canceled)

35. The stabilized food product as in claim 31, wherein the antioxidant compound extends the shelf-life of the food product matrix.

36. (canceled)

37. The stabilized food product as in claim 31, wherein the antioxidant compound is present in the food product matrix at a concentration of less than 3000 ppm.

38.-58. (canceled)

59. A method of preparing a stabilized food product, the method comprising;
adding at least one antioxidant compound of formula II-VII, a salt, or chelate thereof: 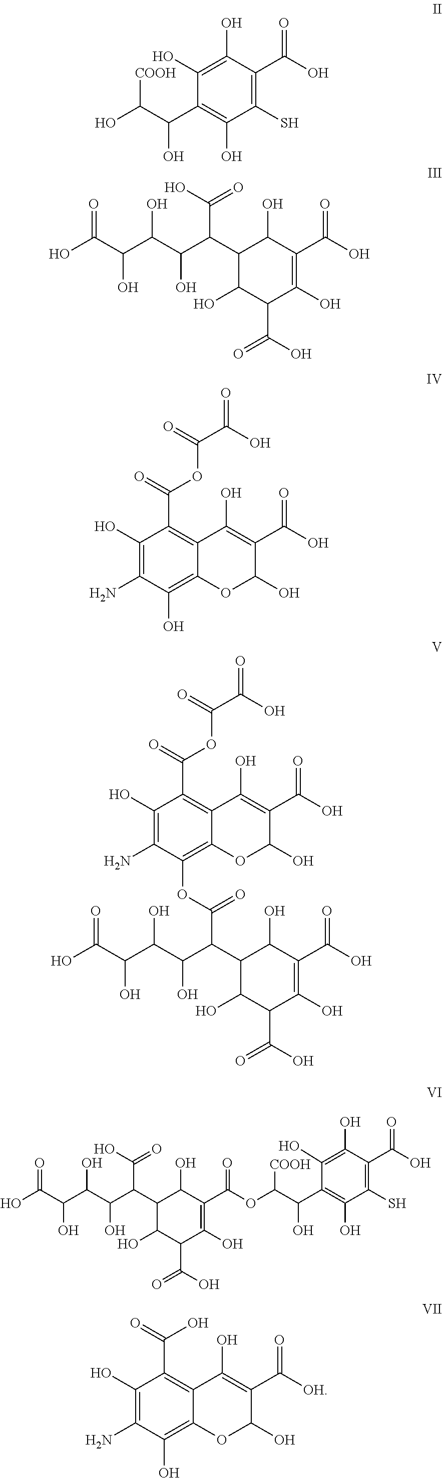
to a food product matrix.

60.-64. (canceled)

65. The method of preparing a stabilized food product as in claim 59, wherein adding the antioxidant compound comprises adding the antioxidant compound to the food product matrix at a concentration of less than 3000 ppm.

66.-73. (canceled)
